(19) United States
(12) Patent Application Publication
(10) Pub. No.: US 2025/0099371 A1
(43) Pub. Date: Mar. 27, 2025

(54) SOLID COMPOSITIONS INCLUDING STABILIZED LIPID NANOPARTICLES AND METHODS OF MAKING MICRONEEDLES COMPRISING SAME

(71) Applicant: Micron Biomedical, Inc., Atlanta, GA (US)

(72) Inventors: Devin V. McAllister, Marietta, GA (US); Marly I. Richter-Roche, Atlanta, GA (US)

(21) Appl. No.: 18/898,285

(22) Filed: Sep. 26, 2024

Related U.S. Application Data
(60) Provisional application No. 63/540,548, filed on Sep. 26, 2023.

(51) Int. Cl. A61K 9/00; A61K 9/51; A61K 47/26; A61K 47/42

(52) U.S. Cl. CPC A61K 9/0021; A61K 9/5123; A61K 47/26; A61K 47/42

(57) ABSTRACT

Microneedle compositions and methods for stabilizing lipid nanoparticle (LNP) formulations containing therapeutic and prophylactic agents are provided. The microneedle compositions, which comprise a gelatin, such as a fish gelatin, are useful in microneedle arrays and microneedle patches for the administration of bioactive agents in LNPs to humans and other subjects.

### CROSS-REFERENCE TO RELATED APPLICATIONS

This application claims priority to U.S. Provisional Application No. 63/540,548, filed on Sep. 26, 2023, which is incorporated by reference herein.

### BACKGROUND

Microneedle arrays represent a cutting-edge technology in drug and vaccine delivery. This drug delivery platform is made from biocompatible materials, which may dissolve upon penetrating the skin, and release their payload directly into the body. This method offers several advantages over traditional injections, including thermostability, reduced pain, improved patient compliance, and in some cases, the elimination of sharps waste. Microneedle arrays can be designed to deliver a wide range of therapeutics, from vaccines to hormones, making them a versatile tool in modern medicine.

Lipid nanoparticles (LNPs) have emerged across the pharmaceutical industry as promising vehicles to deliver various therapeutic agents. Liposomes, an early version of LNPs, are an extremely versatile nanocarrier platform because they can transport hydrophobic or hydrophilic molecules, including small molecules, proteins, and nucleic acids. In fact, liposomes are the earliest nanomedicine delivery platform to successfully proceed from concept to clinical application. A number of liposomal drug formulations have been approved and successfully applied to medical practice.

The next generations of LNPs have been designed with more complex internal architectures and enhanced physical stabilities in addition to the ability to control the location and timing of drug delivery in the body. The use of LNPs in medicine is likely to expand significantly based on recent developments that increase the prospects of their applications. LNPs hold great promise in genetic medicine where gene editing, vaccine development, immuno-oncology, and other genetic therapies rely on the ability to efficiently deliver nucleic acids into cells. Nucleic acid therapeutics are an emerging class of drugs showing potential for treating disease. LNPs have emerged as successful and efficient carriers for such drugs. The successful use of LNPs as a delivery vector for the COVID-19 mRNA vaccines has broadened the horizons for research in mRNA vaccines. The current marketed COVID-19 vaccines are formulated as frozen buffered solutions. While each of these products has been shown to be stable in solution at 4° C. for up to 10 weeks, they are discarded within 24 hours at 25° C. There is also limited information available regarding the stability of LNPs in the solid state.

The stability of LNPs in the solid state is a critical factor for their successful integration with microneedle technology for drug and vaccine delivery. Combining these technologies could potentially revolutionize the field of drug delivery, offering a stable, efficient, and minimally invasive method for administering a wide range of therapeutic agents.

There remains a need for improved stabilization of LNPs in certain solid compositions and for new and improved compositions for forming microneedles that include drug-containing LNP formulations, including but not limited to dissolvable microneedles that include LNPs.

### BRIEF SUMMARY

In one aspect of the disclosure, a solid composition is provided that includes a lipid nanoparticle (LNP) encapsulating a therapeutic agent; and a fish gelatin, and optionally one or more additional pharmaceutically acceptable excipients, such as a carbohydrate. The therapeutic agent may include a nucleic acid, such as an mRNA. The carbohydrate may be methylcellulose, maltodextrin, lactose, trehalose, sucrose, or a combination thereof. The mass ratio of the fish gelatin to the at least one carbohydrate may range from 0.2:1 to 5:1. The solid composition may be formed by casting a liquid comprising (i) the lipid nanoparticle (LNP) encapsulating a therapeutic agent; (ii) the fish gelatin, and (iii) a solvent, and then drying to remove at least enough of the solvent to form the solid composition. The solid composition may be in the form of a microneedle, such as in a microneedle array or microneedle patch. In particular embodiments, the LNP is stabilized in the solid composition. The LNPs may be stabilized in the solid composition and have at least one of, and preferably both, a size range from 200 nm to 400 nm or a polydispersity index (PDI) between 0 and 0.4. In some embodiments, wherein the therapeutic agent comprises a nucleic acid, for example, an mRNA, and the transfection efficiency of the nucleic acid may be at least 25% after storage of the solid composition for three months at a temperature from 2° C. to 8° C.

In another aspect, s microneedle is provided that includes a solid composition comprising: a lipid nanoparticle (LNP) encapsulating a therapeutic agent; and a mammalian gelatin.

In still another aspect, a microneedle array is provided that includes a plurality of microneedles which comprise the solid composition comprising LNPs and gelatin as described above. The microneedle array may be part of a microneedle patch, which may further include a base layer cast onto a base side of the plurality of microneedles.

In a further aspect, a method is provided for making a solid composition, or for stabilizing LNPs in a solid structure, wherein the method includes: (a) preparing a casting liquid which comprises (i) lipid nanoparticle (LNP) encapsulating a therapeutic agent, (ii) a fish gelatin, and (iii) a solvent; (b) casting the casting liquid into or onto a mold; and then (c) drying the cast solution to remove at least enough of the solvent to form the solid composition. The mold may be one that defines an array of microneedles.

In yet another aspect, a method of making a microneedle is provided that includes: (a) preparing a casting liquid which comprises (i) lipid nanoparticle (LNP) encapsulating a therapeutic agent, (ii) a gelatin, and (iii) a solvent; (b) casting the casting liquid into a mold for a microneedle; and then (c) drying the cast solution to remove at least enough of the solvent to form the microneedle. The gelatin may be a fish gelatin, preferably a cold water fish gelatin, or the gelatin may be a mammalian gelatin.

### BRIEF DESCRIPTION OF THE DRAWINGS

The detailed description is set forth with reference to the accompanying drawings. The use of the same reference numerals may indicate similar or identical items. Various embodiments may utilize elements and/or components other than those illustrated in the drawings, and some elements and/or components may not be present in various embodiments. Elements and/or components are not necessarily drawn to scale.

FIG. 1 is a perspective view of a microneedle patch, according to one or more embodiments of the present disclosure.

FIG. 2 is a side view of a microneedle patch, according to one or more embodiments of the present disclosure.

FIG. 3 is a block flow diagram of a method of making a microneedle patch, according to one or more embodiments of the present disclosure.

FIG. 4 is a flow chart illustrating a method of making a microneedle patch, according to one or more embodiments of the present disclosure.

FIG. 5 is a graph comparing the size measurements of lipid nanoparticles stabilized in various excipient formulations, according to one or more embodiments of the present disclosure.

FIG. 6 is a graph comparing the encapsulation efficiency of various excipient formulations, according to one or more embodiments of the present disclosure.

FIG. 7 is a graph comparing the RNA integrity for lipid nanoparticles stabilized in various excipient formulations, according to one or more embodiments of the present disclosure.

FIG. 8 is a graph comparing the relative light units (RLU) for lipid nanoparticles stabilized in various excipient formulations, according to one or more embodiments of the present disclosure.

FIG. 9 is a graph comparing the size measurements of lipid nanoparticles stabilized in various excipient formulations at varying temperatures and concentrations, according to one or more embodiments of the present disclosure.

FIG. 10 is a graph comparing the encapsulation efficiency of various excipient formulations at varying temperatures and concentrations, according to one or more embodiments of the present disclosure.

FIG. 11 is a graph comparing the RNA integrity for lipid nanoparticles stabilized in various excipient formulations at varying temperatures and concentrations, according to one or more embodiments of the present disclosure.

FIG. 12 is a graph comparing the relative light units (RLU) per cell for lipid nanoparticles stabilized in various excipient formulations at varying temperatures and concentrations, according to one or more embodiments of the present disclosure.

FIG. 13 is a graph comparing the size measurements of lipid nanoparticles stabilized in gelatin formulations, according to one or more embodiments of the present disclosure.

FIG. 14 is a graph comparing the encapsulation efficiency of gelatin formulations, according to one or more embodiments of the present disclosure.

FIG. 15 is a graph comparing the RNA integrity for lipid nanoparticles stabilized in gelatin formulations, according to one or more embodiments of the present disclosure.

FIG. 16 is a graph comparing the relative light units (RLU) per cell for lipid nanoparticles stabilized in gelatin formulations, according to one or more embodiments of the present disclosure.

FIG. 17A is a graph comparing the size measurements of lipid nanoparticles stabilized in gelatin formulations, according to one or more embodiments of the present disclosure.

FIG. 17B is a graph comparing the polydispersity index (PDI) for lipid nanoparticles stabilized in gelatin formulations, according to one or more embodiments of the present disclosure.

FIG. 18 is a graph comparing the encapsulation efficiency of gelatin formulations, according to one or more embodiments of the present disclosure.

FIG. 19 is a graph comparing the transfection efficiency for RNA encapsulated in lipid nanoparticles stabilized in gelatin formulations, according to one or more embodiments of the present disclosure.

FIG. 20 is a graph comparing the size measurements of lipid nanoparticles stabilized in gelatin formulations, according to one or more embodiments of the present disclosure.

FIG. 21 is a graph comparing the polydispersity index (PDI) for lipid nanoparticles stabilized in gelatin formulations, according to one or more embodiments of the present disclosure.

FIG. 22 is a graph comparing the encapsulation efficiency of gelatin formulations, according to one or more embodiments of the present disclosure.

FIG. 23 is a graph comparing the transfection efficiency for RNA encapsulated in lipid nanoparticles stabilized in gelatin formulations, according to one or more embodiments of the present disclosure.

### DETAILED DESCRIPTION

Solid compositions and methods for stabilizing lipid nanoparticles (LNP), comprising therapeutic agents, have been developed. It was discovered that gelatin, particularly fish gelatin, can stabilize LNPs in a formulation used to make microneedles, e.g., during a casting and drying process to make the microneedles and subsequently during a storage period needed before the microneedles can be used to administer the therapeutic agent. That is, the solid compositions may be in the form of microneedles, e.g., as microneedle arrays which may be part of a microneedle patch for delivery of therapeutic agents into a subject's skin.

In various embodiments, the mass ratio of gelatin:LNP in the solid composition ranges from 0.1:1 to 20:1, preferably from 0.5:1 to 8:1, and more preferably from 0.5:1 to 4:1. In these embodiments, the gelatin may be fish gelatin, such as cold water fish gelatin, or a mammalian gelatin, such as porcine gelatin.

In a preferred embodiment, the solid composition includes: (i) a lipid nanoparticle (LNP) encapsulating a therapeutic agent; and (ii) a fish gelatin. The composition may include additional excipients with the fish gelatin. In embodiments, the LNPs are dispersed in the fish gelatin and additional excipients. In preferred embodiments, the fish gelatin is effective to stabilize the LNP in the solid composition, such as for at least three months at a temperature from 2° C. to 8° C. In a particular embodiment, microneedles are provided that are formulated with a lipid nanoparticle, an mRNA, gelatin and a carbohydrate such as sugar. The gelatin and sugar can stabilize LNP (which encapsulates the mRNA) during drying, manufacturing and/or storage. In particular embodiments, the gelatin is fish gelatin and the sugar is sucrose and/or trehalose.

In other embodiments, mammalian gelatin, such as porcine gelatin, may be used to form the solid compositions, particularly in the form of microneedles. The compositions may stabilize the LNP.

Compositions and/or formulations comprising a nucleic acid (e.g. an RNA molecule) encapsulated in a lipid nanoparticle (LNP) are provided herein. In some embodiments, the compositions or formulations increase the stability of the nucleic acid, encapsulated in the LNP. In some embodiments, the compositions or formulations described herein are stabilized by the gelatin disclosed herein after a process of drying and storage. For example, such stability can result in retaining structure and/or activity of the nucleic acid after storing the compositions or formulations disclosed herein at a certain temperature for a period of time. In some embodiments, the stability of the nucleic acid, for example mRNA, can be characterized by its transfection efficiency. In some embodiments, the transfection efficiency of the nucleic acid is at least 20%, 25%, 30%, 35%, 40%, 45%, or 50%. In some embodiments, the stability of the nucleic acid, for example mRNA, can be characterized by the particle size of the lipid nanoparticle (LNP). In some embodiments, the particle size of the LNPs is less than 400 nanometers (nm), 350 nm, 300 nm, 250 nm or 200 nm. For example, the particle size of the LNP may be between 200-400 nm. In additional embodiments, the stability of the nucleic acid can be characterized by the polydispersity index (PDI) for lipid nanoparticles. The PDI of the stabilized nucleic acids may be between 0-1, preferably between 0-0.4. In some embodiments, the transfection efficiency, particle size and/or PDI of the nucleic acid is retained in the compositions or formulations described herein after storage at certain temperature for a certain time period. In some embodiments, the compositions or formulations are stabilized at temperatures ranging from about 2° C. to about 40° C., for example, at room temperature, about 20° C. to about 30° C., such as 25° C., or at refrigerated temperatures, for example, 2° C.-8° C. The time period during which the compositions and/or formulations are stored at these temperatures may be at least one day, two days, three days, one week, ten days, two weeks, three weeks, one month, two months, three months, four months, five months, six months, one year, or longer. In some embodiments, transfection efficiency of the nucleic acid of the compositions or formulations disclosed herein is at least 25% after storage at refrigerated temperature for at least three months. In some embodiments, the particle size of the LNP of the compositions or formulations disclosed herein is between 200 nm and 400 nm.

As used herein, the terms “stabilization” and “stability” in reference to LNPs refers to a form in which the LNPs substantially retain their size and form and the biologically active material encapsulated therein essentially retains its physical stability and/or chemical stability and/or biological activity upon storage. Stability can be measured at a selected temperature for a selected period. Trend analysis can be used to estimate an expected shelf life before a material has actually been in storage for that time period.

As used herein, the term “subject” generally refers to humans, but also includes other animals suited for administration of therapeutic agents by microneedles, such as in veterinary or animal husbandry applications.

### Lipid Nanoparticles (LNPs)

A variety of lipid nanoparticles known in the art may be used in the present compositions. Examples include liposomes, solid lipid nanoparticles, nanostructured lipid carriers, cubosomes, cationic lipid-nucleic acid complexes, nucleic acid-carrying LNPs, hybrid lipid-polymeric nanoparticles and nonlamellar lipid nanoparticles. Different lipids have been commonly used to fabricate various lipid-based formulations for the delivery of nucleic acids. Traditional liposomes, lipoplexes, cationic nanoemulsions (CNEs), and nanostructured lipid carriers (NLCs) were developed as delivery systems for nucleic acids.

In various embodiments, the mass ratio of gelatin:LNP in the solid composition ranges from 0.1:1 to 20:1, preferably from 0.5:1 to 8:1, and more preferably from 0.5:1 to 4:1. In some embodiments, the mass ratio of the gelatin:LNP is 0.5:1; 1:1; 1.5:1, 2:1, 2.5:1, 3:1, 3.5:1, 4:1, 4.5:1 or 5:1. In particular embodiments the mass ratio of gelatin:carbohydrate is 4:1 or between the range of 3:1 to 5:1. The mass of the LNP in the mass ratios provided is exclusive of the therapeutic agent it encapsulates.

The LNP may be at least one of: a liposome, solid lipid nanoparticle (SLN), a nanostructured lipid carrier (NLC), a cubosome, cationic lipid-nucleic acid complexes, nucleic acid-carrying lipid nanoparticles hybrid lipid-polymeric nanoparticles and/or nonlamellar lipid nanoparticles. In some embodiments, the LNP may be between 100 to 300 nm in diameter.

Liposomes, an early version of LNPs, are versatile nanocarrier platforms because they can transport hydrophobic or hydrophilic molecules, including small molecules, proteins, and nucleic acids. Solid lipid nanoparticles (SLNs) have a solid lipid core and are typically used for certain types of drug delivery applications, such as controlled release and improved stability. NLCs are composed of a mixture of solid and liquid lipids, which form a stable matrix that can encapsulate drugs.

Nucleic acid-carrying lipid nanoparticles have been investigated and successfully entered into the clinic for the delivery of small molecules, siRNA drugs and mRNA. The two authorized COVID-19 mRNA vaccines, mRNA-1273 and BNT162b, use lipid nanoparticles to deliver antigen mRNA. Many other lipid nanoparticle-mRNA formulations have been developed and are under clinical evaluation for the prevention and treatment of virus infections, cancer and genetic diseases.

Nucleic acid-carrying LNPs can be used to encapsulate nucleic acids. The nucleic acid-carrying LNPs are a specific type of lipid nanoparticle designed to encapsulate and deliver nucleic acids, such as mRNA. In one embodiment, the LNP can be an mRNA-carrying LNP. These LNPs can be composed of several types of lipids, including: ionizable lipids, pegylated lipids, phospholipids and cholesterol. These lipid nanoparticles protect the mRNA from degradation and facilitate its delivery into cells.

Cationic lipids—ALC-0315 (Pfizer) and SM-102 (Moderna) are used in the COVID-19 vaccine nanoparticles; both lipids are tertiary amines which are protonated (and thus positively charged) at low pH. Their hydrocarbon chains are connected through biodegradable ester groups, enabling safe clearance after mRNA delivery. The cationic lipids used in the mRNA vaccines contain branched hydrocarbon chains which optimize the formation of nonlamellar phases and the mRNA delivery efficiency. The PEG-lipids are both PEG-2000 conjugates. The LNPs are prepared at low pH (pH 4.0), at which the ionizable lipid is positively charged, so that it can easily form complexes with mRNA. A microfluidic device may be used to mix a stream containing mRNA in water with a stream containing a lipid mixture in ethanol. When rapidly mixed, the constituents of these two streams form nanoparticles which entrap the negatively charged mRNA.

TABLE 1
Lipid Constituents of the LNP
Carriers of COVID-19 mRNA Vaccines

| | | | |
|---|---|---|---|
| | | | CAS |
| | | Abbreviation | Registry |
| Lipid | Purpose | or Lab Code | Number |

BNT162b2 vaccine (Pfizer/BioNTech)

| | | | |
|---|---|---|---|
| (4-hydroxybutyl)azanediyl | ionizable | ALC-0315 | 2036272- |
| bis(hexane-6,1-diyl)bis(2- | cationic | | 55-4 |
| hexyldecanoate) | lipid | | |
| (2-hexyldecanoate), | PEG-lipid | ALC-0159 | 1849616- |
| 2-[(polyethylene | | | 42-7 |
| glycol)-2000]-N,N- | | | |
| ditetradecylacetamide | | | |
| 1,2-distearoyl-sn-glycero-3- | helper lipid | DSPC | 816-94-4 |
| phosphocholine | | | |
| cholesterol | helper lipid | Chol | 57-88-5 |

mRNA-1273 vaccine (Moderna)

| | | | |
|---|---|---|---|
| heptadecan-9-yl 8-((2- | ionizable | SM-102 | 2089251- |
| hydroxyethyl)(6-oxo-6- | cationic | | 47-6 |
| (undecyloxy)hexyl)amino)octanoate | lipid | | |
| 1,2-dimyristoyl-rac-glycero-3- | PEG-lipid | PEG2000- | 160743- |
| methoxypolyethylene glycol-2000 | | DMG | 62-4 |
| 1,2-distearoyl-sn-glycero-3- | helper lipid | DSPC | 816-94-4 |
| phosphocholine | | | |
| cholesterol | helper lipid | Chol | 57-88-5 |

### Gelatin

Suitable gelatins known in the art may be used in the compositions and methods described herein. The gelatin may be fish gelatin or porcine gelatin, for example. In one embodiment, the fish gelatin is a cold water fish gelatin.

The stabilization of the LNP in the solid microneedle formulation is provided by combining the LNP formulation with gelatin. Gelatin is a water-soluble protein extracted from animal tissue and used as a stabilizer. The melting and gelling temperatures of gelatin play a crucial role in stabilizing pharmaceuticals. Gelatin begins to gel as it cools at specific temperatures. This gelling process creates a semi-solid matrix that can encapsulate and protect LNPs that contain active pharmaceutical ingredients (APIs). The gelatin can protect LNPs from environmental factors such as light, oxygen, and moisture. Gelatin's melting point is usually in the range 28-31° C. for mammal-derived gelatin and in the range of 11-28° C. for fish-derived gelatin. Generally, the gel strength, gelling, and melting points of mammalian-derived gelatins are much higher than those of fish-derived gelatins. Indeed, the typical gel strength, gelling point, and melting point temperatures for mammalian gelatins are found in the range 100-300 grams (Bloom), 20-25° C. and 28-31° C., respectively, in comparison to fish gelatins' values, which are about 70-270 grams (Bloom), 8-25° C. and 11-28° C., respectively. Bloom values for gelatin are given in grams. This measurement indicates the force required to depress a standard plunger into the gelatin gel by a specific distance, reflecting the gel's strength.

In some embodiments, the gelatin has a Bloom value of 70-270 grams. In some embodiments, the gelatin has a gelling point of 2-25° C. In one embodiment, the gelatin has a Bloom value of 50-125 grams. In another embodiment, the gelatin has a gelling point of 2-8° C. In some embodiments, the gelatin has a melting point of 11-28° C. In some embodiments, the gelatin is fish gelatin, including but not limited to cold water fish gelatin.

In some embodiment, the gelatin has a Bloom value of 100-300 grams. In some embodiments, the gelatin has a gelling point of 20 to 25° C. In one embodiment, the gelatin has a Bloom value of 250-300 grams and a gelling point of 23-25° C. In some embodiments, the gelatin has a melting point of 28-31° C. In some embodiments, the gelatin is a mammalian gelatin, including but not limited to porcine gelatin.

Gelatin's rheological properties (gel strength, viscosity) and thermal stability (melting and gelling temperature) define it, in addition to basic physico-chemical characteristics (solubility, composition, transparency, color, smell, and taste). Gel strength is defined by the so-called “Bloom value”, and it was found to decrease when the pH value was below 5 and above 9, while it remained almost constant in the pH range 5-9, with some variations. Thus, the determination of the Bloom force is usually performed at pH values from 4.6 to 7.0. Another important physical property of gelatin is its viscosity, which depends on concentration, temperature, and pH. Indeed, viscosity was found to increase with polymer concentration and decrease with temperature and pH. Gelatin's thermal stability is another important parameter that is influenced by several parameters such as polymer concentration, molecular weight distribution, and Bloom value. Gelatin's gelling time has also been analyzed. Its thermo-reversible gelation mechanism has been extensively studied. It is known that, at low temperatures, gelatin chains undergo a conformational disordered-ordered transition and are able to form thermo-reversible networks by the formation of hydrogen bonds. In particular, gelatins are found in the solid state at high temperatures (>40° C.) as single coils. Above a determined critical concentration (usually about 1%), they are able to assemble into thermo-reversible gels with a disordered organization when the temperature is cooled down below 30° C.

Gelatin's advantages include low cost, easy availability, biodegradability, and low immunogenicity, high biocompatibility and intrinsic bioactivity.

The gelatin used in the compositions and methods disclosed herein can be a gelatin suitable for use as a pharmaceutical excipient. In some embodiments, the gelatin is a gelatin that can stabilize a vaccine and protect the vaccine's active ingredients from adverse conditions such as freeze-drying or heat. In some embodiments, the gelatin is porcine gelatin. The porcine gelatin can be the porcine gelatin used in the following vaccines: MMR (Measles, Mumps, Rubella), Varicella (Chickenpox), Yellow Fever, Live, Attenuated Influenza (Flumist). In some other embodiments, bovine gelatin can be used such as the bovine gelatin used in the rabies vaccine, Rabies (Rabavert).

In some particular embodiments, fish gelatin is used in the compositions and methods described herein. Fish gelatin is derived from the collagen found in the skin, bones, scales, and fins of various fish species. The fish gelatin can be selected from but not limited to the following: Cold-Water Fish Gelatin, Alaska Cod, Pacific Cod, Green Pollock, Salmon, Warm-Water Fish Gelatin, Tilapia, Grass Carp, Squid and/or tuna fish gelatin. In a preferred embodiment, cold water fish gelatin is used.

### Other Matrix Components

In addition to the gelatin and LNPs, the solid composition includes one or more other components forming the matrix of the solid composition (e.g., the microneedle composition) in which the LNP encapsulating a therapeutic agent are dispersed and stabilized.

In a preferred embodiment, these other components include at least one carbohydrate, such as a disaccharide or polysaccharide. For example, the at least one carbohydrate may be selected from methylcellulose, maltodextrin, lactose, trehalose, sucrose, and combination thereof. In a preferred embodiment, the at least one carbohydrate is a sugar, such as sucrose and/or trehalose.

In various embodiments, the mass ratio of gelatin:carbohydrate in the solid composition ranges from 0.1:1 to 20:1, preferably from 0.2:1 to 5:1, and more preferably from 0.5:1 to 2:1. In some embodiments, the ratio of the gelatin:carbohydrate is 0.5:1; 1:1; 1.5:1, 2:1, 2.5:1 or 3:1. In particular embodiments the ratio of gelatin:carbohydrate is 1:1 or 2:1 or between the range of 0.5:1 to 2.5:1.

Other suitable sugars that may be used in the solid compositions and microneedles described herein include fructose, galactitol, glucose, mannitol, mannose, sorbitol, xylitol, xylose, lactose, maltose, pullulan and/or chitosan, instead of or in combination with sucrose and/or trehalose.

Other water-soluble excipients may be used in the solid compositions and microneedles described herein include dextran, maltodextrin, hyaluronic acid, caboxymethyl cellulose Na, methylcellulose, hydroxypropyl cellulose, hydroxypropyl methylcellulose, poly(2-ethyl-2-oxazoline), polyacrylic acid sodium salt, polyacrylic acid.

In some embodiments, the solid composition may include 30% by weight sucrose and 70% by weight gelatin, or 70% by weight sucrose and 30% by weight gelatin. In some embodiments, the microneedle formulation includes 1 to 50% by weight mRNA encapsulated in a LNP, 10-50% by weight gelatin, and 25-70% by weight sugar.

In some embodiments, microneedles are formed of or coated with a composition that includes an mRNA as the therapeutic agent, which is encapsulated in lipid nanoparticles, and an excipient formulation that includes a gelatin and a sugar, which respectively may be a fish gelatin and sucrose. The LNPs are dispersed in the excipient formulation. In some embodiments, the ratio of the gelatin to sugar is 0.5:1; 1:1; 1.5:1, 2:1, 2.5:1 or 3:1. For example, in certain embodiments, the ratio of gelatin to sugar may be 1:1 or 2:1. In some embodiments, the excipient may include one or more additional ingredients in addition to the sugar and gelatin, where the one or more additional ingredients may be trehalose. In a preferred embodiment, the water-soluble polymer is poly(vinyl alcohol) (PVA).

In some embodiments, the matrix material includes one or more of the following biodegradable polymers: polylactic acid (PLA), polyglycolic acid (PGA), polylactive co-glycolic acid (PLGA), and/or polyethylene glycol (PEG, PEG 300, PEG 400, PEG 600, PEG 3350, PEG 4000), poly(lactide)s, poly(glycolide)s, poly(lactide-co-glycolide)s, polyanhydrides, polyorthoesters, polyetheresters, polycarpolactones, polyesteramides, poly(butyric acid)s, poly(valeric acid)s, polyhydroxyalkanoates, degradable polyurethanes, any copolymers thereof, and any blends thereof.

### Therapeutic Agent

As used herein, the term “therapeutic agent” refers to any therapeutic, prophylactic, or diagnostic substance suitable for administration to a biological tissue such as the skin of a subject, such as for medical applications. In various embodiments, the mass ratio of gelatin:therapeutic agent ranges from 1:1 to 200:1, preferably from 10:1 to 100:1, and more preferably from 40:1 to 80:1. In particular embodiments, the mass ratio of gelatin:therapeutic agent can be 20:1, 30:1 40:1, 50:1, 60:1, 70:1, 80:1, 90:1, 100:1 or 120:1. In these embodiments, the gelatin may be fish gelatin, such as cold water fish gelatin, or a mammalian gelatin, such as porcine gelatin.

In some embodiments, the therapeutic agent is selected from small molecules and larger biotechnology produced or purified molecules (e.g., peptides, proteins, DNA, RNA). In some embodiments, the therapeutic agent comprises a nucleic acid. In some embodiments, the therapeutic agent comprises mRNA.

In certain embodiments, the mass ratio of gelatin:mRNA ranges from 1:1 to 200:1, preferably from 10:1 to 100:1, and more preferably from 40:1 to 80:1. In particular embodiments, the mass ratio of gelatin:mRNA is 20:1, 30:1 40:1, 50:1, 60:1, 70:1, 80:1, 90:1, 100:1 or 120:1. In these embodiments, the gelatin may be fish gelatin, such as cold water fish gelatin, or a mammalian gelatin, such as porcine gelatin.

In some embodiments, therapeutic agent is a vaccine. Non-limiting examples of vaccines include those known in the art for the prevention of coronavirus (COVID-19) anthrax, cervical cancer (human papillomavirus), dengue fever, diphtheria, Ebola, hepatitis A, hepatitis B, hepatitis C, haemophilus influenza type b (Hib), HIV/AIDS, human papillomavirus (HPV), influenza (seasonal and pandemic), lyme disease, malaria, measles, meningococcal, monkeypox, mumps, pertussis, pneumococcal, polio, rabies, rotavirus, rubella, shingles (herpes zoster), smallpox, tetanus, typhoid, tuberculosis (TB), varicella (chickenpox), West Nile, and yellow fever.

In some other embodiments, the therapeutic agent is one known or found to be useful in the treatment of these or other diseases and ailments, including but not limited to suitable small molecule pharmaceutically active ingredients, for systemic, regional or local administration via a microneedle patch, applied to a subject's skin or other tissue sites.

### Microneedle Arrays and Patches

The solid compositions described herein can be used to make a variety of microneedle devices. The composition may form the structure of at least the tip portions of the microneedles in an array of a microneedle patch.

In some embodiments, microneedle arrays include a base layer and an array of microneedles extending from the base layer, where at least a distal tip portion of each of the microneedles contains the LNPs and are configured to be inserted into the tissue of a subject. The microneedles may also include a drug-free, or substantially drug-free, proximal portion between the base layer and the distal tip portions of the microneedles.

In some embodiments, a microneedle patch is provided that includes at least one microneedle array that includes the solid compositions described herein. The solid composition may form all or a portion of each of the microneedles, e.g., the distal tip portion of the microneedle, or a coating on a microneedle substrate made of another material. The microneedle patch may further include a base layer, which may be cast onto a base side (e.g., at the proximal end) of the plurality of microneedles. The base layer may be made of a water insoluble material, such as a biodegradable polymer. It has been found that the base layer may promote or enhance the stability of the LNPs in the microneedle.

In a preferred embodiment, microneedles are provided that are formed of a solid composition that includes: (i) a lipid nanoparticle (LNP) encapsulating a therapeutic agent; (ii) a fish gelatin, wherein the mass ratio of fish gelatin:LNP ranges from 0.5:1 to 4:1; and (iii) sucrose, wherein the mass ratio of fish gelatin:sucrose ranges from 0.5:1 to 2:1. In some of these embodiments, the therapeutic agent includes mRNA and the mass ratio of fish gelatin:mRNA ranges from 40:1 to 80:1.

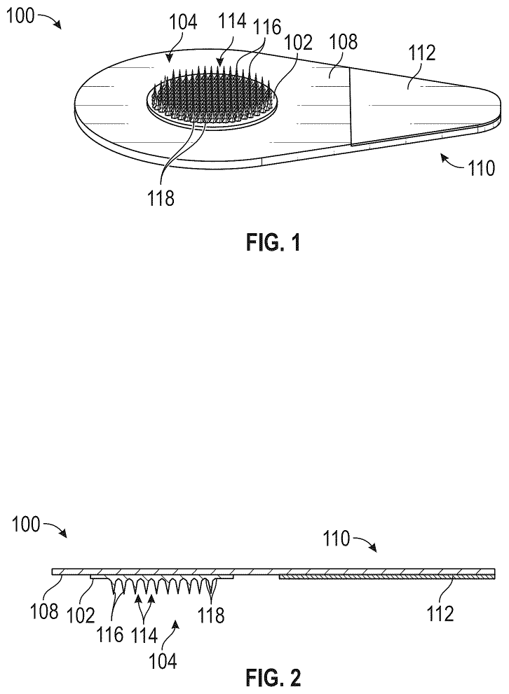

FIGS. 1-2 show one example of a microneedle array 104 as part of a microneedle patch 100, where each microneedle 114 has a tip portion 116 that extends from a funnel portion 118. The microneedle array 104 includes a base layer 102 from which the microneedles extend. The funnel portions 118 extend from a microneedle side of the base layer 102. The microneedle array 104 may be affixed to a handling layer 108 by an adhesive layer (not shown) disposed therebetween. The handling layer 108 includes a tab portion 110 that extends away from the microneedle array 104. The tab portion 110 enables a person to hold and manipulate the microneedle patch 100 without having to contact the microneedles 114. An adhesive cover 112 may also be affixed to the portion of the adhesive layer that overlays the tab portion 110 of the handling layer 108. The adhesive cover 112 enables a person to hold and manipulate the microneedle patch 100 without having to contact the adhesive layer. An optional force feedback indicator (not shown) may also be disposed between the adhesive layer and the handling layer 108. Some examples of suitable force feedback indicators for microneedle patches are described in U.S. Pat. No. 10,265,511, which is incorporated herein by reference.

Examples of suitable microneedles and array structures that can be made with the solid formulations described herein are disclosed in U.S. Pat. No. 10,828,478, which is incorporated herein by reference.

The microneedle array may include a single therapeutic agent, or it may have two or more different therapeutic agents. The therapeutic agent can be embedded within a singular excipient formulation, or within different excipient formulations. For example, the microneedles can be formulated with different excipient formulations to obtain the desired stability and/or release kinetics. In some embodiments, the microneedle patch can contain microneedles that are made of different matrix materials arranged within the same microneedle. For example, the tip of the microneedle may be formed of a water-soluble matrix containing the therapeutic agent (e.g., mRNA), and the proximal portion of the microneedle may be formed of a biodegradable polymer (different from the water-soluble matrix).

In some embodiments, the distal tip portions of each microneedle of the microneedle arrays is configured to separate from at least the base layer in less than 5 minutes, preferably less than one minute. In some embodiments, the microneedles are dissolvable. For example, the tip portions of the microneedles may be dissolvable, while the proximal portion and/or funnel portion, if included, is not dissolvable. In some embodiments, dissolvable microneedles may degrade within the tissue into which they were inserted over an extended period of time. For example, after separation from the base, microneedle may degrade in vivo over 1 day to 24 weeks.

Dissolvable microneedles may be formulated with water-soluble excipients, including any such excipients known in the art. For example, the water-soluble excipient may a combination of a sugar or sugar alcohol, a water-soluble polymer, or a combination thereof. The water-soluble material may include, but is not limited to, dextran, natural polysaccharides, hyaluronic acid, chitosan, beta-sodium glycerophosphate, hydroxypropyl beta cyclodextrin and/or water-soluble polymers, such as poly(vinyl alcohol) (PVA), polyvinyl pyrrolidone (PVP). The water-soluble matrix may also include additional excipients, including but not limited to (i) dextrose, maltose, sorbitol, glycerol, glucose, xylitol, or a combination thereof, (ii) glycerol and/or (iii) a buffer, such as HEPES, phosphate buffer, Tris/HCL, potassium phosphate, ammonium acetate, or a combination thereof.

The microneedle array described above may be combined with one or more other components to produce a microneedle patch, such as a patch that can be manually applied to the skin of a patient. For example, the microneedle array may be combined with an adhesive layer, which may be used to facilitate securing the patch to a patient's skin during the period of administration of the substance of interest. A backing or handle layer may further be included to facilitate handling of the patch, as described and illustrated in FIGS. 1-2.

The backing layer may be made out of a variety of different materials, and may be the same or different than the tab portion. In some embodiments, the backing layer may be a composite material or multilayer material including materials with various properties to provide the desired properties and functions. For example, the backing material may be flexible, semi-rigid, or rigid, depending on the particular application. As another example, the backing layer may be substantially impermeable, protecting the one or more microneedles (or other components, from moisture, gases, and contaminants. Alternatively, the backing layer may have other degrees of permeability and/or porosity based on the desired level of protection. Non-limiting examples of materials that may be used for the backing layer include various polymers, elastomers, foams, paper-based materials, foil-based materials, metallized films, and non-woven and woven materials.

### Methods of Making the Solid Composition and Microneedles

The solid compositions and microneedles described herein may be made by any suitable method. In some embodiments, the methods described herein are used to make microneedle arrays, which, generally described, include a base substrate with a plurality of microneedles extending from the base substrate. The process entails providing a suitable mold, filing the mold with suitable fluidized materials, drying or curing the fluidized materials to form the microneedles, any funnel portions (if included between the base substrate and the microneedles), and the base substrate, and then removing the formed part from the mold. These filing and drying steps may be referred to herein as “casting.” Examples of suitable methods are described in U.S. Pat. No. 10,828,478, which is incorporated herein by reference.

In some embodiments, a method of making a solid composition is provided that includes the following steps: (a) preparing a casting liquid which comprises (i) lipid nanoparticle (LNP) encapsulating a therapeutic agent, (ii) a fish gelatin, and (iii) a solvent; (b) casting the casting liquid into or onto a mold; and then (c) drying the cast liquid to remove at least enough of the solvent to form the solid composition. The mold may be a mold defining an array of microneedles, such that the solid composition produced by the method is in the form of a plurality of microneedles.

In some embodiments, a method of making a microneedle is provided that includes the following steps: (a) preparing a casting liquid which comprises (i) lipid nanoparticle (LNP) encapsulating a therapeutic agent, (ii) a gelatin, and (iii) a solvent; (b) casting the casting liquid into a mold for a microneedle; and then (c) drying the cast liquid to remove at least enough of the solvent to form the microneedle.

The casting liquid for use in any of these methods may be prepared by combining selected amounts of the LNPs, which have a desired type and amount of therapeutic agent encapsulated therein, with the gelatin, solvent, and one or more other pharmaceutically acceptable excipients. The “casting liquid” may be an aqueous solution with LNPs dispersed/suspended therein. In some embodiments, the therapeutic agent includes an mRNA. In some embodiments, the solvent comprises water. In some embodiments, the solvent can include Tris. In some embodiments, the casting liquid is from 1 wt % to 10 wt % gelatin, preferably from 4 wt % to 8 wt % of gelatin. In some embodiments, the gelatin preferably is a fish gelatin, such as a cold water fish gelatin. In some embodiments, the other excipients are selected to form a microneedle with the required mechanical properties (e.g., stiffness) to be effective to be insertable into skin, e.g., to penetrate the stratum corneum, or other tissues. In some embodiments, the additional pharmaceutically acceptable excipient included in the casting liquid includes a carbohydrate, such as a disaccharide or polysaccharide. For example, the carbohydrate may be methylcellulose, maltodextrin, lactose, trehalose, sucrose, and a combination thereof.

Any suitable means for drying the cast solution may be used. As used herein, the term “drying,” “dried,” or “dry” as it refers to the material in a mold refers to the material becoming at least partially solidified. The microneedles may be removed from the mold before being fully dried or after the microneedles are dried to an operational state. In some preferred embodiments, the drying is performed at a temperature from 4° C. to 40° C., preferably from 4° C. to 10° C. After drying, the solid formulation, or microneedle, is removed from the mold.

In a preferred embodiment, a two-step filling process is used to make microneedles, wherein the first filling step contains the casting liquid comprising the LNPs (with therapeutic agent) and gelatin, which substantially migrates into the microneedle tips during the drying process. This is followed by a second filling step and a subsequent drying or curing step. This second filling step contains the matrix material(s) that forms the base of the microneedles and funnels, if included, and may be overfilled to create the base substrate or part of the base substrate.

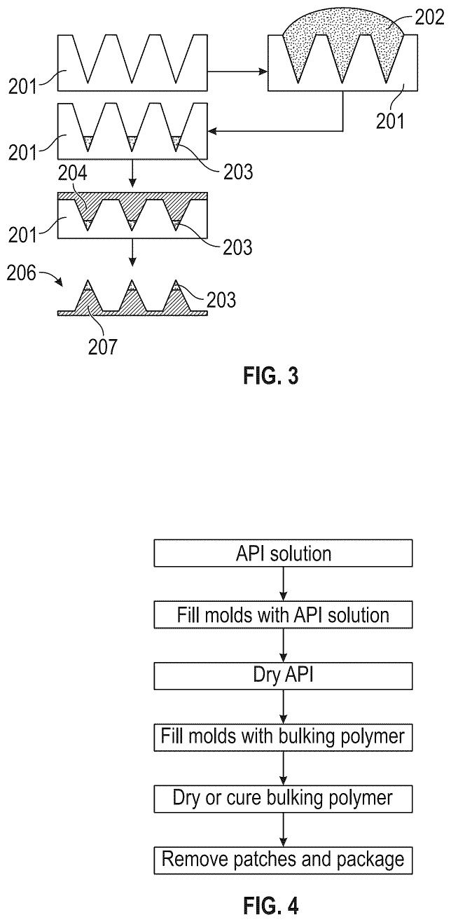

FIG. 3 illustrates one embodiment of a molding process that includes two castings. In this method, a mold 201 is provided and then filled with a first casting liquid 202 and then dried, thereby forming distal tip portions 203 of the microneedles of a microneedle array 206. After which, the mold 201 is filled with a second casting liquid 204 and then dried or cured, thereby forming a corresponding proximal portion 207 for each microneedle of the microneedle array 206. The microneedle array 206 is then removed from the mold 201.

In a preferred embodiment, the first casting liquid includes the LNPs (with therapeutic agent) and gelatin, and one or more suitable water soluble excipients, and the second casting liquid comprises one or more different components, not including the LNPs, therapeutic agent, and gelatin. Instead, the second casting liquid may include one or more a water soluble or non water soluble polymers, or a polymerizable (curable) precursor. The second casting liquid may include a suitable aqueous or organic solvent. The second casting liquid optionally may include a second, different therapeutic agent.

A process flow diagram of this method illustrated in FIG. 3 is also illustrated in the block flow diagram shown in FIG. 4.

In a preferred embodiment, the therapeutic agent is loaded preferentially into the microneedles and their tips, and not into the proximal portions of the microneedles and funnel portions if provided.

In some other embodiments, a single filling step or more than two filling steps may be used. A single filling step may be desirable, for example, if the therapeutic agent is inexpensive and the excess therapeutic agent can be wasted. More than two filling steps may be desirable to further increase the loading of the therapeutic agent within the microneedle tips, deposit multiple therapeutic agent or excipients within different microneedles or sections of microneedles within a given microneedle patch, and/or impart further functionality into the microneedle patch.

The microneedle patches may be inspected and packaged to protect it from mechanical damage, moisture, light, oxygen, and/or microbial or other contamination before it can be used.

### Methods of Using the Microneedle Arrays

The microneedle arrays and patches provided herein may be self-administered or administered by another individual (e.g., a parent, guardian, minimally trained healthcare worker, expertly trained healthcare worker, and/or others). In some embodiments, the microneedle patches described herein are used to deliver the therapeutic agent into the skin by inserting the microneedles across the stratum corneum (outer 10 to 20 microns of skin that is the barrier to transdermal transport) and into the viable epidermis and dermis. The small size of the microneedles enables them to cause little to no pain and target the intradermal space. The intradermal space is highly vascularized and rich in immune cells and provides an attractive path to administer both vaccines and therapeutics.

In a preferred embodiment, the microneedles are dissolvable and once in the intradermal space they dissolve within the interstitial fluid and release the therapeutic agent into the skin. Once the microneedles are fully dissolved, which may take less than 20 minutes, the patch backing can be removed and discarded as non-sharps waste since the microneedles have dissolved away. The microneedles can be altered to provide for more rapid release or quicker separation from the patch backing. They can also be formulated to release the therapeutic agent over an extended period. Alternatively, the microneedles can be designed to rapidly separate from the patch, but then either dissolve or biodegrade to release the therapeutic agent after the insertion and separation. A combination of these release features may be contained within a single microneedle patch to provide the desired release profile of the therapeutic agent.

### EXAMPLES

The present invention may be further understood with reference to the following non-limiting examples.

### Example 1. Initial Excipient Screen

Several excipient formulations (Table 1) were assessed for use as stabilizing excipients for lipid nanoparticles encapsulating mRNA, for formulation into a microneedle patch. Samples were prepared as films and as dissolvable microneedle patches (dMPs). Film samples contained 100 mcg mRNA and 2 mg lipid nanoparticles (LNPs). To create the film samples, 100 μL of each sample formulation, detailed in Table 1, was deposited onto a substrate and dried at ambient conditions overnight.

dMP samples contained 50 mcg mRNA and 1 mg LNPs. Exemplary microneedle patches were formulated, using the sample formulations, according to the methods described herein. A 40 μL deposit of each sample formulation was deposited onto a suitable microneedle array mold and was centrifuged at 4° C. and 3000×g for one hour. After centrifugation, 150 μL of a second cast solution containing 18 wt % PVA/18 wt % sucrose in water was deposited onto the dried sample material within the mold.

TABLE 2
Initial Excipient Screen Sample Descriptions.

| Sample Number | Sample Description |
|---|---|
| 1 | mRNA-LNP/4% sucrose |
| 2 | mRNA-LNP/4% sucrose/5% polyvinyl alcohol (PVA) |
| 3 | mRNA-LNP/4% sucrose/5% povidone (PVP) |
| 4 | mRNA-LNP/4% sucrose/5% gelatin |
| 5 | mRNA-LNP/4% sucrose/5% lactose |
| 6 | mRNA-LNP/4% sucrose/5% maltodextrin |
| 7 | mRNA-LNP/4% sucrose/1% methylcellulose |
| 8 | mRNA-LNP |
| 9 | mRNA-LNP/4% PVA |
| 10 | mRNA-LNP/4% PVP |
| 11 | mRNA-LNP/4% gelatin |
| 12 | mRNA-LNP/4% lactose |
| 13 | mRNA-LNP/4% maltodextrin |
| 14 | mRNA-LNP/4% hyaluronic acid |
| 15 | mRNA-LNP/4% PVP/1% maltodextrin |
| 16 | mRNA-LNP/4% PVP/1% sucrose |
| 17 | mRNA-LNP/4% PVP/1% lactose |
| 18 | mRNA-LNP/4% gelatin/1% sucrose |
| 19 | mRNA-LNP/4% gelatin/1% lactose |
| 20 | mRNA-LNP-dMP 5% gelatin |
| 21 | mRNA-LNP-dMP 3.75% hyaluronic acid |

All percentages are by weight (wt %) in aqueous solution.

In general, all LNP particle diameters increased for the tested samples, as compared to a control. Most samples were also multimodal. Encapsulation efficiency, integrity, and expression levels were also low as compared to the control samples.

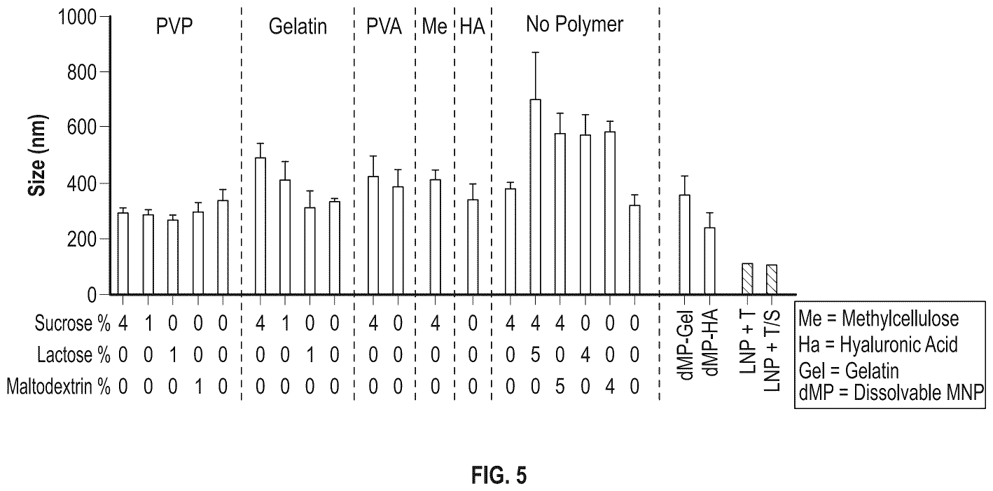

Size measurements using dynamic light scattering (DLS) were taken, the results of which are shown in FIG. 5. A large increase in particle diameter size was observed. All polydispersity index (PDI) measurements were multimodal except the controls LNP+Tris (T)/S and LNP+T with PDIs of 0.206 and 0.137, respectively.

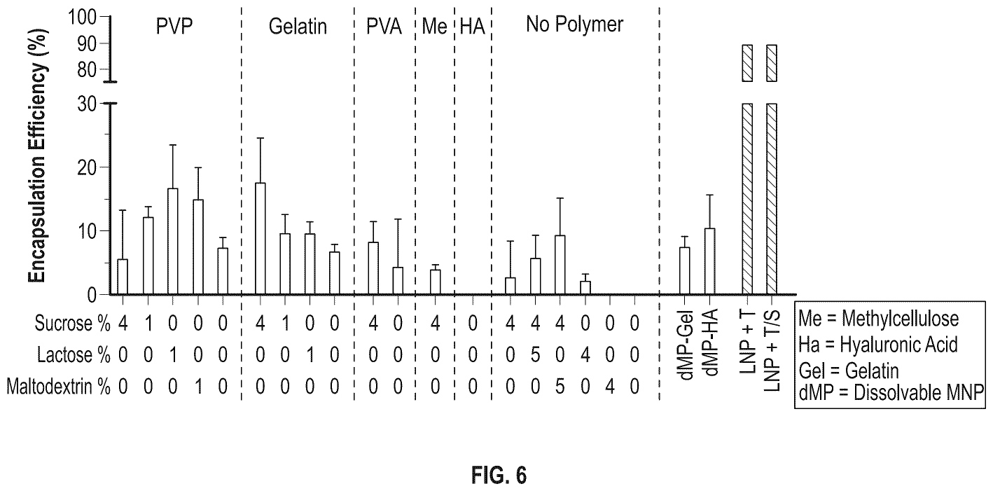

All samples also displayed significant loss in mRNA encapsulation efficiency, as shown in FIG. 6.

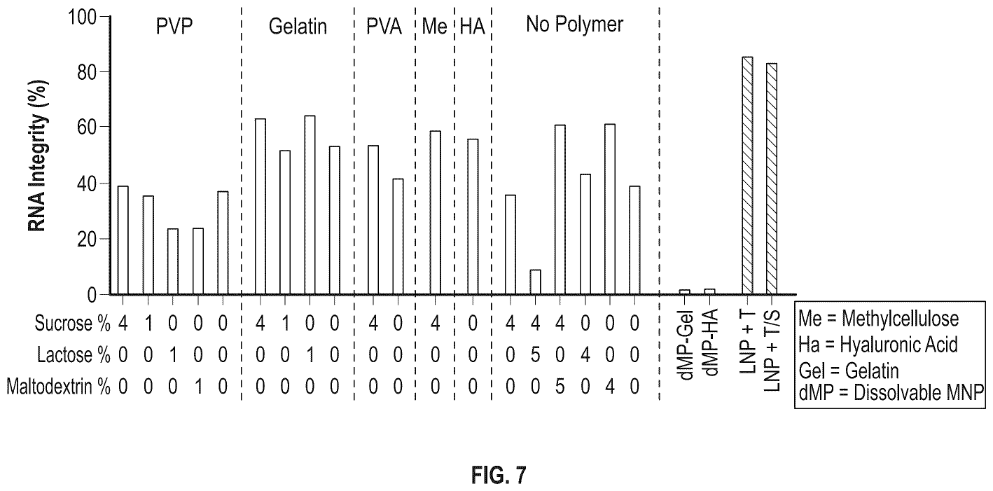

Similarly, samples also resulted in greater loss of intact mRNA as compared to the controls, as shown in FIG. 7. Replicate No. 1 from each sample was analyzed for mRNA integrity.

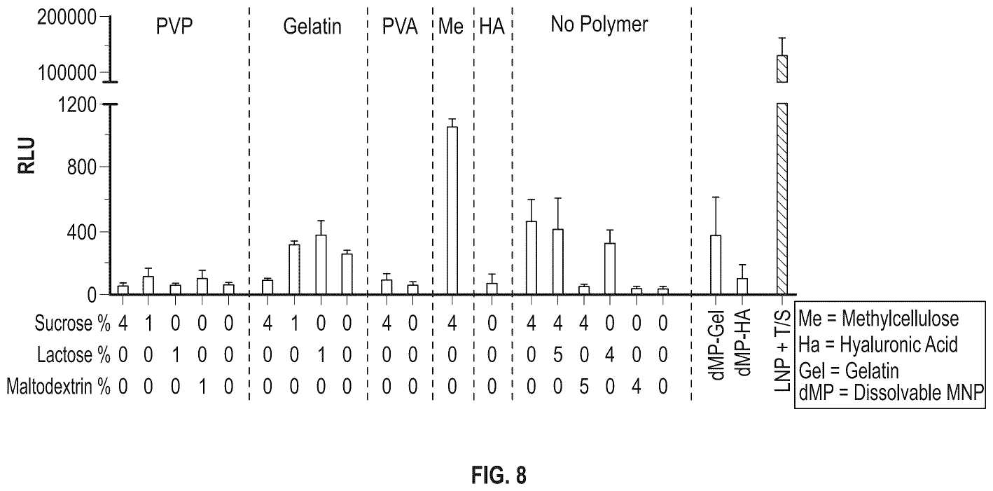

FIG. 8 demonstrates that samples showed significantly lower expression in cells compared to the LNP control. All samples had minimal expression in HeLa cell transfection (background=approx. 100-200 relative light units (RLU, a measure of transfection of the luciferase gene). The data is from a repeat transfection due to instrument software malfunction. The results were not significantly different from the original transfection.

### Example 2. Secondary Excipient Screen

Samples having higher excipient contents, as compared to those described in Example 1, were assessed for use as stabilizing excipients for lipid nanoparticles encapsulating mRNA, for formulation into a microneedle patch. The mRNA volume and drying time and temperature were also varied for said samples, as detailed in Table 3.

TABLE 3
Secondary Excipient Screen Sample Descriptions.

| Sam- | | Drying | |
|---|---|---|---|
| ple | | Temper- | |
| Num- | | ature | Dose |
| ber | Sample Description | (° C.) | (mg) |
| 1 | mRNA-LNP/8% sucrose | 4 | 0.01 |
| 2 | mRNA-LNP/40% sucrose | 4 | 0.01 |
| 3 | mRNA-LNP/8% sucrose/8% trehalose | 4 | 0.01 |
| 4 | mRNA-LNP/8% sucrose/8% maltodextrin | 4 | 0.01 |
| 5 | mRNA-LNP/8% sucrose/8% gelatin | 4 | 0.01 |
| 6 | mRNA-LNP/8% sucrose | 4 | 0.02 |
| 7 | mRNA-LNP/8% sucrose | 4 | 0.1 |
| 8 | mRNA-LNP/40% sucrose | 4 | 0.1 |
| 9 | mRNA-LNP/8% sucrose/8% trehalose | 4 | 0.1 |
| 10 | mRNA-LNP/8% sucrose/8% maltodextrin | 4 | 0.1 |
| 11 | mRNA-LNP/8% sucrose/8% gelatin | 4 | 0.1 |
| 12 | mRNA-LNP/8% sucrose | 25 | 0.01 |
| 13 | mRNA-LNP/40% sucrose | 25 | 0.01 |
| 14 | mRNA-LNP/8% sucrose/8% trehalose | 25 | 0.01 |
| 15 | mRNA-LNP/8% sucrose/8% maltodextrin | 25 | 0.01 |
| 16 | mRNA-LNP/8% sucrose/8% gelatin | 25 | 0.01 |
| 17 | mRNA-LNP/8% sucrose | 25 | 0.1 |
| 18 | mRNA-LNP/40% sucrose | 25 | 0.1 |
| 19 | mRNA-LNP/8% sucrose/8% trehalose | 25 | 0.1 |
| 20 | mRNA-LNP/8% sucrose/8% maltodextrin | 25 | 0.1 |
| 21 | mRNA-LNP/8% sucrose/8% gelatin | 25 | 0.1 |
| 22 | mRNA-LNP/8% sucrose | 40 | 0.01 |
| 23 | mRNA-LNP/40% sucrose | 40 | 0.01 |
| 24 | mRNA-LNP/8% sucrose/8% trehalose | 40 | 0.01 |
| 25 | mRNA-LNP/8% sucrose/8% maltodextrin | 40 | 0.01 |
| 26 | mRNA-LNP/8% sucrose/8% gelatin | 40 | 0.01 |
| 27 | ff_{LUC} LNP (+) Control | n/a | n/a |

All percentages are by weight (wt%) in aqueous solution.

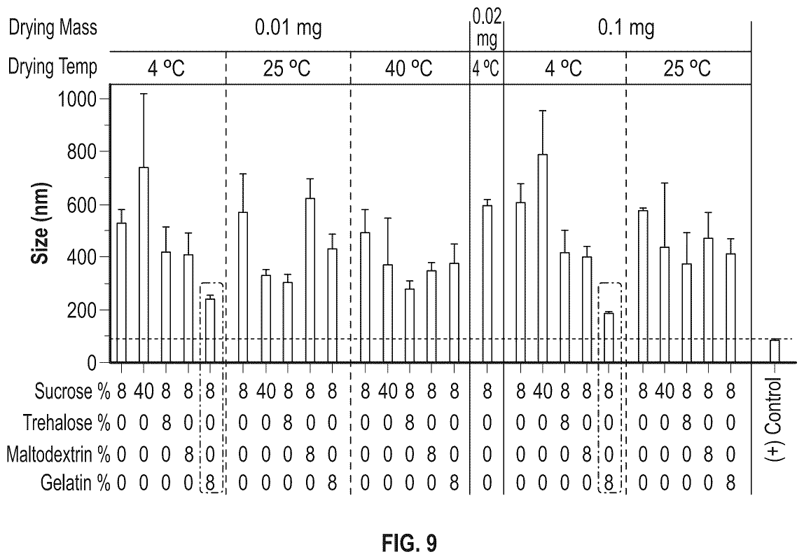

Particle size was measured using DLS, and as shown in FIG. 9, particle sizes increased for all samples relative to the control. Most PDIs were also multimodal or greater than 0.4, except samples with gelatin. Samples 5 and 11 containing gelatin were less than 300 nm in size. The positive control sample had a PDI of about 0.1.

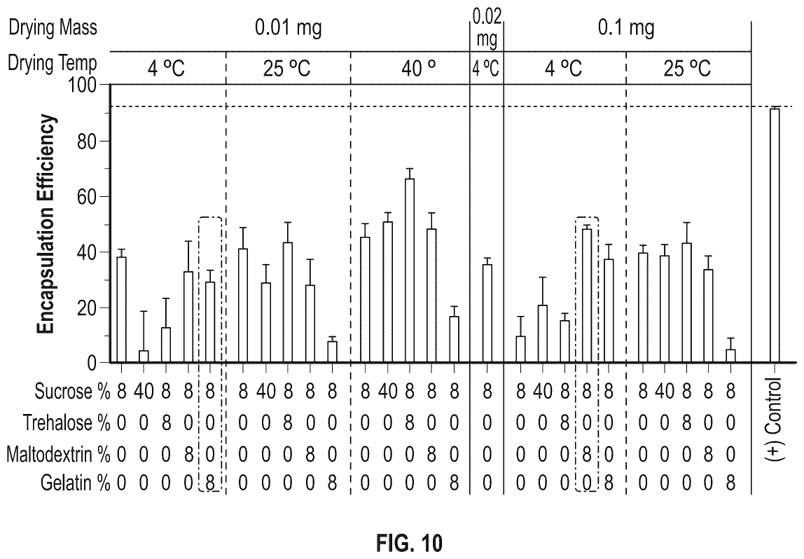

As shown in FIG. 10, a loss in encapsulation efficiency was also observed in all samples. Most encapsulation efficiency was below 50%.

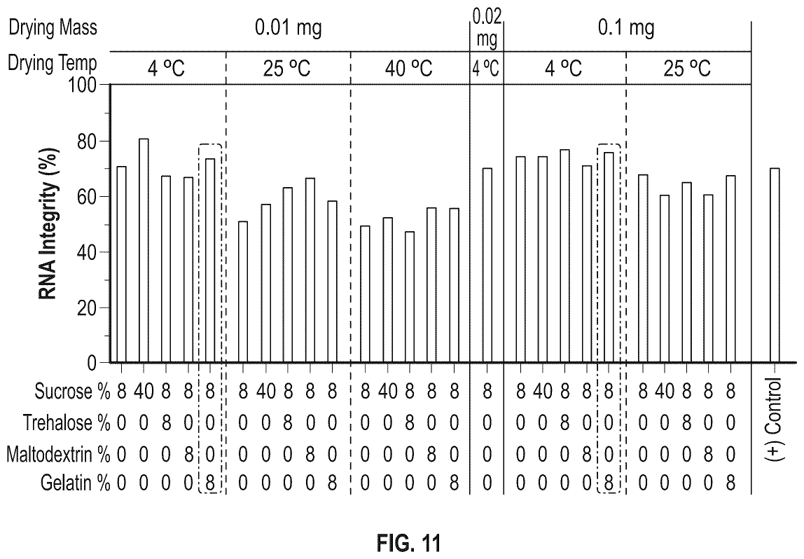

FIG. 11, however, shows that minimal change in RNA purity was observed at lower drying temperatures, and was generally comparable to the positive control. Some loss of RNA purity was seen with higher drying temperatures.

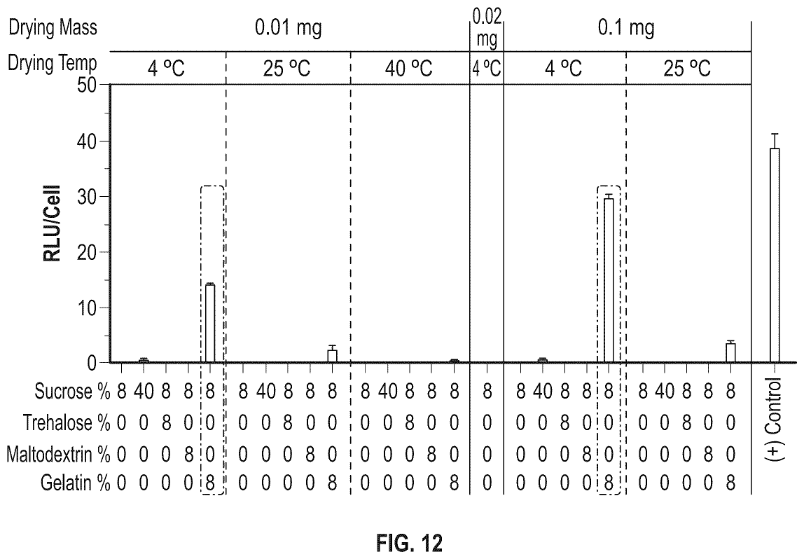

FIG. 12 shows samples dried in gelatin (Samples 5, 11, 16, 21, and 26) at lower temperatures exhibited successful transfection of mRNA. Samples dried in gelatin at 4° C. (Samples 5 and 11) led to optimal transfection. Some signal loss was detected with samples dried at 25° C. with gelatin (Samples 16 and 21).

### Example 3. Tertiary Excipient Screen

A third excipient screen was conducted to evaluate higher excipient contents, gelatin types and proteins, drying temperatures, as shown in Table 4. Samples dried at 40° C. were dried for approximately 7 hours, and samples dried at 4° C. were dried for 2 days. Film and microneedle patch samples were formed according to the methods described with respect to Example 1.

TABLE 4
Third Excipient Screen Sample Descriptions

| Sam- | | Drying | |
|---|---|---|---|
| ple | | Temper- | |
| Num- | | ature | Dose |
| ber | Sample Description | (° C.) | (mg) |
| 1 | mRNA-LNP/8% sucrose/8% fish gelatin | 4 | 0.1 |
| 2 | mRNA-LNP/4% sucrose/4% fish gelatin | 4 | 0.1 |
| 3 | mRNA-LNP/8% sucrose/4% fish gelatin | 4 | 0.1 |
| 4 | mRNA-LNP/4% sucrose/8% fish gelatin | 4 | 0.1 |
| 5 | mRNA-LNP/8% sucrose/8% BSA | 4 | 0.1 |
| 6 | mRNA-LNP/8% sucrose/8% trehalose | 4 | 0.1 |
| 7 | mRNA-LNP/8% sucrose/4% fish | 4 | 0.1 |
| | gelatin/4% trehalose | | |
| 8 | mRNA-LNP/8% sucrose/4% porcine | 4 | 0.1 |
| | gelatin | | |
| 9 | mRNA-LNP/4% sucrose/4% porcine | 4 | 0.1 |
| | gelatin | | |
| 10 | mRNA-LNP/8% sucrose/1% porcine | 4 | 0.1 |
| | gelatin | | |
| 11 | mRNA-LNP/8% sucrose/8% fish gelatin | 40 | 0.1 |
| 12 | mRNA-LNP/4% sucrose/4% fish gelatin | 40 | 0.1 |
| 13 | mRNA-LNP/8% sucrose/4% fish gelatin | 40 | 0.1 |
| 14 | mRNA-LNP/4% sucrose/8% fish gelatin | 40 | 0.1 |
| 15 | mRNA-LNP/8% sucrose/8% BSA | 40 | 0.1 |
| 16 | mRNA-LNP/8% sucrose/8% trehalose | 40 | 0.1 |
| 17 | mRNA-LNP/8% sucrose/4% fish | 40 | 0.1 |
| | gelatin/4% trehalose | | |
| 18 | mRNA-LNP/8% sucrose/4% porcine | 40 | 0.1 |
| | gelatin | | |
| 19 | mRNA-LNP/4% sucrose/4% porcine | 40 | 0.1 |
| | gelatin | | |
| 20 | mRNA-LNP/8% sucrose/1% porcine | 40 | 0.1 |
| | gelatin | | |
| 21 | dMP-1 | | 0.04 |
| 22 | ff_{LUC} (+) Control | n/a | n/a |

All percentages are by weight (wt %) in aqueous solution.

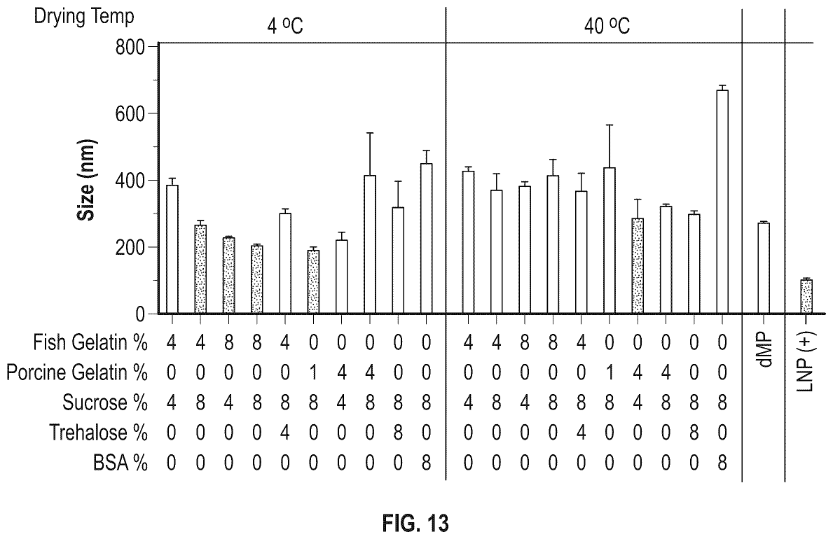

FIG. 13 shows that particle size, measured by DLS, increased as compared to the control for all samples. The shaded bar indicates samples having a non-multimodal size distribution. Samples dried in a high percentage of fish gelatin and sucrose at 4° C. had the least size growth and greatest prevalence of non-multimodal distribution.

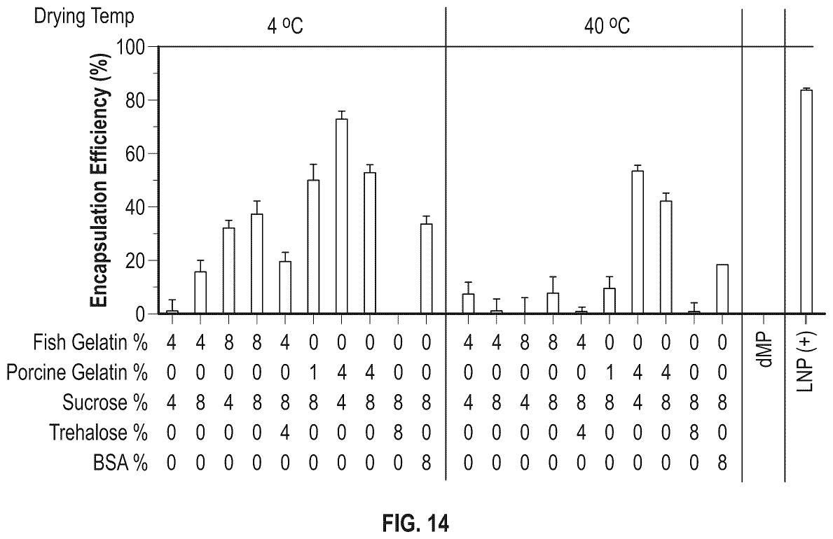

As shown in FIG. 14, samples dried at 40° C. exhibited higher loss of mRNA encapsulation compared to those dried at 4° C. Samples dried in porcine gelatin at 4° C. had the highest encapsulation. Porcine gelatin samples were the only samples with significant entrapment at 40° C. The dMP sample had no measured entrapment of mRNA.

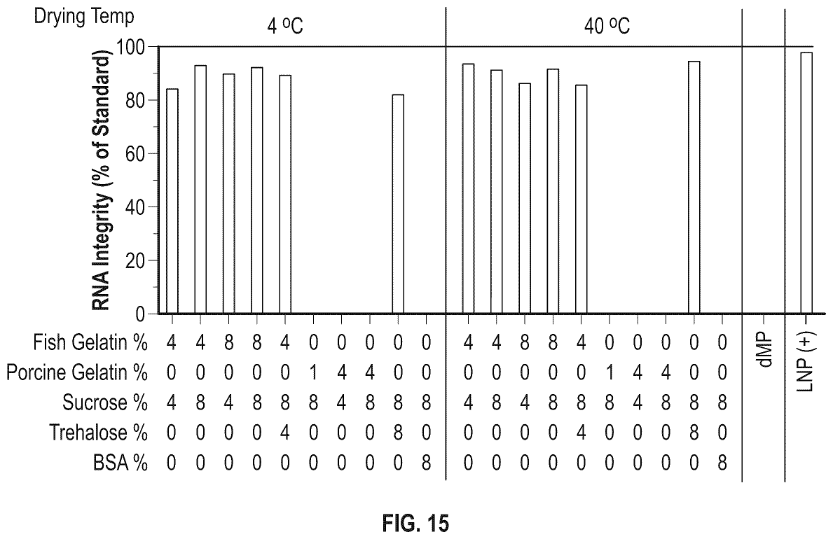

As shown in FIG. 15, RNA purity was generally high as a fraction of an mRNA standard. The RNA purity assay was not successful for porcine gelatin samples, BSA samples, or the dMP sample. This is likely due to excipient materials interfering with the assay. Fish gelatin samples may also require further assay development for more reliable analysis.

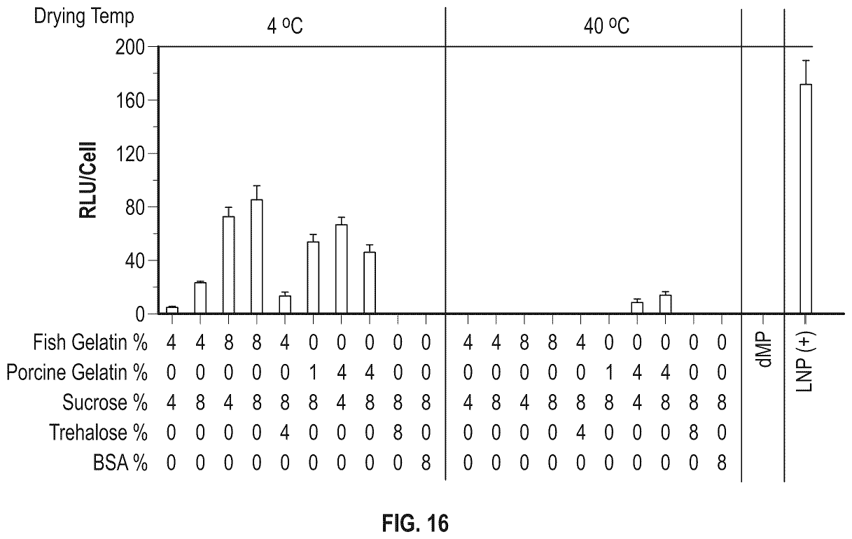

FIG. 16 demonstrates that samples dried in gelatin at lower temperatures exhibited successful transfection. Higher percentage of fish gelatin led to higher levels of transfection. Porcine gelatin samples also exhibited transfection at 4° C. and 40° C., but was lower at 40° C. The dMP sample did not exhibit successful transfection.

Samples having gelatin and sucrose dried at 4° C. showed positive results, with the particle size measuring greater than 300 nm and having the lowest PDI values. These samples also had the highest level of mRNA expression in HeLa cells. These positive results were observed for the fish and porcine gelatin samples.

### Example 4. Fish Gelatin Screen

Formulations of 8% sucrose/8% fish gelatin dMP samples were evaluated with either a dissolving second (ND) cast (PVA/sucrose in water) or non-dissolving second casts selected from Polycaprolactone (PCL) (ND1), two-part urethane (ND2), or biomed clear resin (ND3), as shown in Table 5. Film samples (F) were also prepared according to the method described in Example 1.

TABLE 5
Sucrose/Gelatin Sample Descriptions

| | | | Dose |
|---|---|---|---|
| Sample | Format | Sample Description | (mg) |
| D (Dissolving) 1 | Microneedle | mRNA-LNP in 8% sucrose/8% | 0.04 |
| | Array | | |
| | (MNA) | fish gelatin | |
| ND (Non- | MNA | mRNA-LNP in 8% sucrose/8% | 0.04 |
| dissolving) 1 | | fish gelatin | |
| ND2 | MNA | mRNA-LNP in 8% sucrose/8% | 0.04 |
| | | fish gelatin | |
| ND3 | MNA | mRNA-LNP in 8% sucrose/8% | 0.04 |
| | | fish gelatin | |
| F (Film) 1 | Film (F) | mRNA-LNP in 8% sucrose/8% | 0.04 |
| | | fish gelatin | |
| F2 | F | mRNA-LNP in 8% sucrose/8% | 0.1 |
| | | fish gelatin | |
| (+) | | ffLuc LNP (+) Control | n/a |

All percentages are by weight (wt %) in aqueous solution.

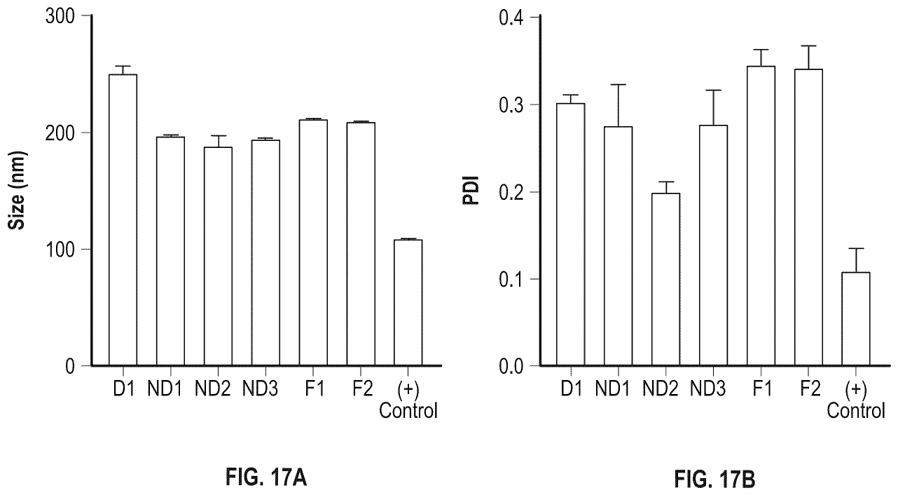

FIGS. 17A-17B demonstrate that LNP particles, measured by DLS, increased in size and PDI compared to the positive control for all samples. Non-multimodal, but still high, PDIs were also observed for all samples. The dissolvable patch (Sample D1) had a larger particle size as compared to the non-dissolvable patch samples, with Sample ND2 having the lowest PDI of the samples tested.

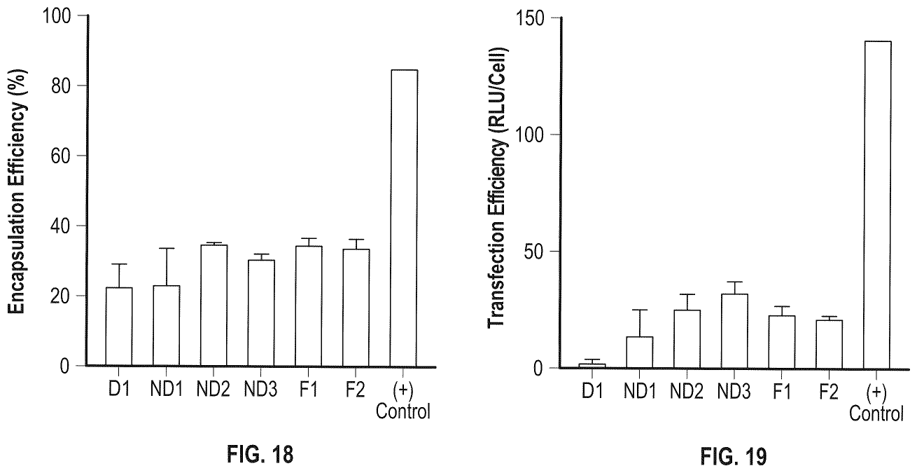

FIG. 18 shows that all samples exhibited reduction in mRNA encapsulation as compared to the control sample, with values ranging between 20% and 40%. There was no significant difference observed between the film samples and the patch samples. Sample ND1 exhibited a significant loss of mRNA; however this is likely attributable to a procedural error.

FIG. 19 indicates that the non-dissolvable patch samples and film samples demonstrated successful transfection of mRNA. The dissolvable patch sample did not exhibit successful transfection, which is likely attributable to a procedural error. Film samples performed comparably to the non-dissolvable patch samples.

A few microneedle patches formulated from the sample formulations were also assessed for mechanical strength The characteristics of the microneedle patches can be summarized as follows. On average individual needles from Sample D1 had the highest overall mechanical strength under compression compared to the other microneedle patches. Across the patches tested, on average, the buckling points identified from the curves were higher than ultimate rupture stress of human skin; however, most variants of the patches did have microneedles that failed before this point. These data were variable which indicates that more microneedles would need to be compressed to get mechanical strength under axial compression.

Microneedles from Sample D1 were considered ductile when compared to the other patches. Samples ND1-ND3 were more brittle underaxial compression in comparison to Sample D1.

Almost all microneedles from Sample D1 were intact upon visual inspection prior to testing with few showing tip damage, however, microneedles from Samples ND1-ND3 showed a large percentage of microneedles significantly damaged under visual inspection, which made mechanical testing difficult. Some of the damage may be attributed to potential damage during handling however some of the damage observed is likely attributed to poor molding.

Based on the data that was collected (mechanical strength and visual inspection via Keyence microscope), Sample D1 performed best.

### Example 5. Secondary Fish Gelatin Screen

Lead microneedle array formulation samples that were fabricated by depositing/casting & drying an aqueous formulation containing mRNA LNPs and 8 wt % sucrose and 8 wt % fish gelatin into microneedle molds followed by the addition of a non-dissolvable array base layer were stored for 3 months refrigerated (2-8° C.). The 40 μL deposition volume included 0.04 mg of mRNA and 0.8 mg of LNPs. The second material cast, that formed the non-dissolving base of the microneedle array, included polycaprolactone (PCL) (ND1, ND4) a biocompatible and low melting point polymer that was deposited as a heated melt that solidifies as it cooled down to ambient temperature, two-part urethane (from Smooth-On, Inc, Macungie, PA) (ND2 and ND6-ND9) that cured within a few minutes (note, ND7 and ND8 contained 10% and 25% ethanol in the first cast solution containing the LNPs, and Biomed Clear resin (by FormLabs Somerville, MA) (ND3 and ND5) that was cured via exposure to visible light, e.g., 405 nm. Table 6 describes the samples.

TABLE 6
Sucrose/Gelatin Sample Descriptions.

| Sample | | | Dose |
|---|---|---|---|
| Number | Format | Sample Description | (mg) |
| ND (non | Microneedle | mRNA-LNP in 8% sucrose/8% | 0.04 |
| dissolving) 1 | array (MNA) | fish gelatin | |
| ND2 | MNA | mRNA-LNP in 8% sucrose/8% | 0.04 |
| | | fish gelatin | |
| Film (F) | Film (F) | mRNA-LNP in 8% sucrose/8% | 0.04 |
| | | fish gelatin | |
| ND4 | MNA | mRNA-LNP in 8% sucrose/8% | 0.04 |
| | | fish gelatin | |
| ND5 | MNA | mRNA-LNP in 8% sucrose/8% | 0.04 |
| | | fish gelatin | |
| ND6 | MNA | mRNA-LNP in 8% | 0.04 |
| | | sucrose/8% fish gelatin | |
| ND7 | MNA | mRNA-LNPin 8% sucrose/8% | 0.04 |
| | | fish gelatin | |
| ND8 | MNA | mRNA-LNP in 8% sucrose/8% | 0.04 |
| | | fish gelatin | |
| ND9 | MNA | mRNA-LNP in 4% sucrose/4% | 0.04 |
| | | lactose/8% fish gelatin | |
| (+) | | ffLuc (+) Control | n/a |

All percentages are by weight (wt %) in aqueous solution.

Only three of the formulation samples (ND1, ND2, and F) were stored refrigerated for 3 months. The other samples were freshly prepared before testing.

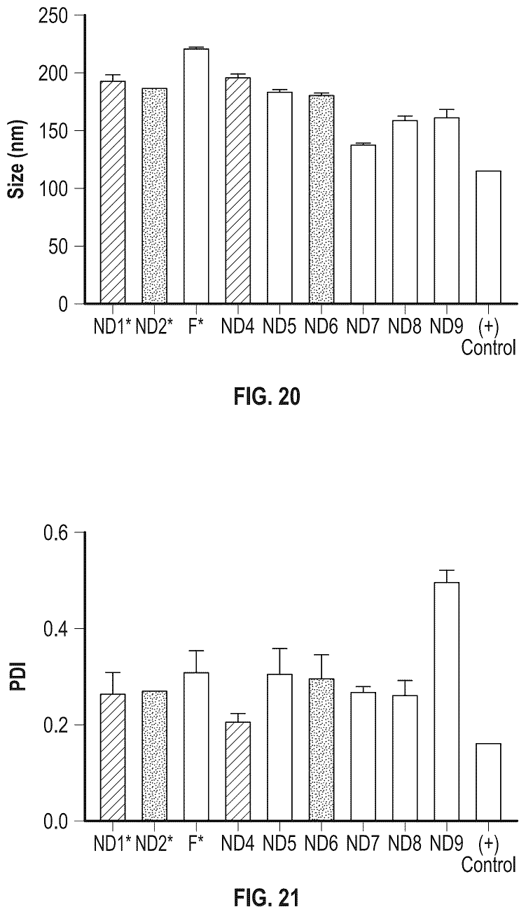

FIGS. 20-21 show that particle size and PDI, measured by DLS, increased for all samples as compared to the positive control. Non-multimodal, but elevated, PDI was also observed for all samples. All particle size and PDI measurements were within an acceptable range.

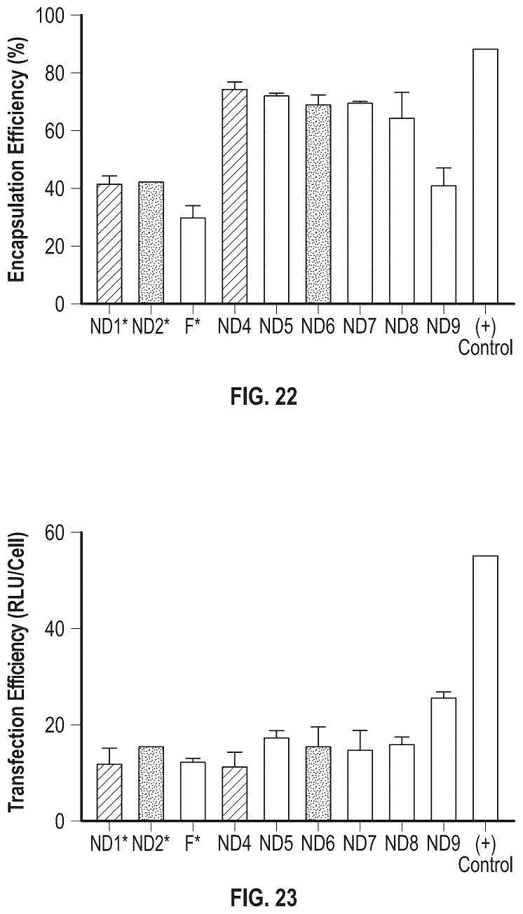

As shown in FIG. 22, mRNA encapsulation was reduced for all samples as compared to the positive control. However, the samples demonstrated similar encapsulation compared to the samples of Example 4, which were not subjected to prolonged storage. Samples ND4-ND8 exhibited unexpectedly high encapsulation of mRNA.

FIG. 23 demonstrates that the samples exhibited successful mRNA transfection. All samples had successful transfection of HeLa cells. Reduced transfection was observed as compared to those of Example 4, but notably the control sample also exhibited lower transfection.

These formulation screening data indicates that cold water fish gelatin works to stabilize LNPs during microneedle patch manufacturing and storage, and data for samples stored for three months is similar to that of fresh samples. mRNA-dMPs after refrigerated storage for 3 months are similar to fresh samples. Microneedle patches with a non-dissolving second cast performed better than those with a dissolving second cast, independent of the non-dissolving second cast material.

### EXEMPLARY EMBODIMENTS

Embodiment 1. A solid composition comprising: a lipid nanoparticle (LNP) encapsulating a therapeutic agent; and a fish gelatin.

Embodiment 2. The solid composition of Embodiment 1, wherein the fish gelatin is a cold water fish gelatin.

Embodiment 3. The solid composition of Embodiment 1 or 2, wherein the mass ratio of the fish gelatin to the LNP ranges from 0.5:1 to 8:1.

Embodiment 4. The solid composition of Embodiment 1 or 2, wherein the mass ratio of fish gelatin:LNP ranges from 0.5:1 to 4:1.

Embodiment 5. The solid composition of any one of Embodiments 1 to 4, further comprising one or more additional pharmaceutically acceptable excipients.

Embodiment 6. The solid composition of any one of Embodiments 1 to 5, wherein the therapeutic agent comprises mRNA.

Embodiment 7. The solid composition of any one of Embodiments 1 to 6, wherein the solid composition further comprises at least one carbohydrate.

Embodiment 8. The solid composition of Embodiment 7, wherein the at least one carbohydrate is a disaccharide.

Embodiment 9. The solid composition of Embodiment 7, wherein the at least one carbohydrate is a polysaccharide.

Embodiment 10. The solid composition of Embodiment 7, wherein the at least one carbohydrate is selected from the group consisting of methylcellulose, maltodextrin, lactose, trehalose, sucrose, and combination thereof.

Embodiment 11. The solid composition of Embodiment 7, wherein the at least one carbohydrate is sucrose.

Embodiment 12. The solid composition of any one of Embodiments 7 to 11, wherein the mass ratio of the fish gelatin to the at least one carbohydrate ranges from 0.2:1 to 5:1.

Embodiment 13. The solid composition of any one of Embodiments 7 to 11, wherein the mass ratio of the fish gelatin to the at least one carbohydrate ranges from 0.5:1 to 2:1.

Embodiment 14. The solid composition of any one of Embodiments 1 to 13, wherein the solid composition is formed by casting a liquid comprising (i) the lipid nanoparticle (LNP) encapsulating a therapeutic agent; (ii) the fish gelatin, and (iii) a solvent, and then drying to remove at least enough of the solvent to form the solid composition.

Embodiment 15. The solid composition of Embodiment 14, wherein the drying is performed with the liquid at a temperature from 4° C. to 40° C., preferably from 4° C. to 10° C.

Embodiment 16. The solid composition of any one of Embodiments 1 to 15, wherein the solid composition in the form of a microneedle.

Embodiment 17. The solid composition of any one of Embodiments 1 to 15, wherein the solid composition in the form a coating on a microneedle.

Embodiment 18. The solid composition of any one of Embodiments 1 to 17, wherein the LNP is stabilized in the solid composition, preferably for at least three months at a temperature from 2° C. to 8° C.

Embodiment 19. The solid composition of any one of Embodiments 1 to 18, wherein the LNPs are stabilized in the solid composition and have at least one of, and preferably both, a size range from 200 nm to 400 nm or a polydispersity index (PDI) between 0 and 0.4.

Embodiment 20. The solid composition of any one of Embodiments 1 to 19, wherein the therapeutic agent comprises a nucleic acid, for example, an mRNA, and the transfection efficiency of the nucleic acid is at least 25% after storage of the solid composition for three months at a temperature from 2° C. to 8° C.

Embodiment 21. A microneedle array comprising a plurality of microneedles which comprise the solid composition of any one of Embodiments 1 to 20.

Embodiment 22. A microneedle patch comprising at least one microneedle array of Embodiment 21.

Embodiment 23. The microneedle patch of Embodiment 22, further comprising a base layer cast onto a base side of the plurality of microneedles.

Embodiment 24. The microneedle patch of Embodiment 23, wherein the base layer comprises a water insoluble material.

Embodiment 25. A microneedle formed of a solid composition comprising: a lipid nanoparticle (LNP) encapsulating a therapeutic agent; a fish gelatin, wherein the mass ratio of fish gelatin:LNP ranges from 0.5:1 to 4:1; and sucrose, wherein the mass ratio of fish gelatin:sucrose ranges from 0.5:1 to 2:1.

Embodiment 26. The microneedle of Embodiment 25, wherein the therapeutic agent comprises mRNA and the mass ratio of fish gelatin:mRNA ranges from 40:1 to 80:1.

Embodiment 27. The microneedle of Embodiment 25 or 26, wherein the LNP is stabilized in the solid composition, preferably for at least three months at a temperature from 2° C. to 8° C.

Embodiment 28. A microneedle comprising a solid composition comprising: a lipid nanoparticle (LNP) encapsulating a therapeutic agent; and a mammalian gelatin.

Embodiment 29. A method of making a solid composition, the method comprising: preparing a casting liquid which comprises (i) lipid nanoparticle (LNP) encapsulating a therapeutic agent, (ii) a fish gelatin, and (iii) a solvent; casting the casting liquid into or onto a mold; and then drying the cast solution to remove at least enough of the solvent to form the solid composition.

Embodiment 30. The method of Embodiment 29, wherein the drying is performed with the liquid at a temperature from 4° C. to 40° C., preferably from 4° C. to 10° C.

Embodiment 31. The method of Embodiment 29 or 30, wherein the casting liquid is cast into a mold defining an array of microneedles.

Embodiment 32. The method of any one of Embodiments 29 to 31, wherein the casting liquid comprises from 1 wt % to 10 wt % of the fish gelatin, preferably from 4 wt % to 8 wt % of the fish gelatin.

Embodiment 33. The method of any one of Embodiments 29 to 32, wherein the fish gelatin is a cold water fish gelatin.

Embodiment 34. The method of any one of Embodiments 29 to 33, wherein the mass ratio of fish gelatin:LNP in the solid composition ranges from 0.5:1 to 8:1, preferably from 0.5:1 to 4:1.

Embodiment 35. The method of any one of Embodiments 29 to 34, wherein the therapeutic agent comprises mRNA.

Embodiment 36. The method of any one of Embodiments 29 to 35, wherein the mass ratio of fish gelatin:therapeutic agent in the solid composition ranges from 10:1 to 100:1, preferably from 40:1 to 80:1.

Embodiment 37. The method of any one of Embodiments 29 to 36, wherein the casting liquid further comprises one or more additional pharmaceutically acceptable excipients.

Embodiment 38. The method of Embodiment 37, wherein the one or more additional pharmaceutically acceptable excipients comprises a carbohydrate, which optionally is a disaccharide or polysaccharide.

Embodiment 39. The method of Embodiment 38, wherein the carbohydrate is selected from the group consisting of methylcellulose, maltodextrin, lactose, trehalose, sucrose, and combination thereof.

Embodiment 40. The method of Embodiment 39, wherein the carbohydrate comprises sucrose.

Embodiment 41. The method of any one of Embodiments 38 to 40, wherein the mass ratio of the fish gelatin to the carbohydrate in the solid composition ranges from 0.2:1 to 5:1, preferably from 0.5:1 to 2:1.

Embodiment 42. A method of making a microneedle, the method comprising:
preparing a casting liquid which comprises (i) lipid nanoparticle (LNP) encapsulating a therapeutic agent, (ii) a gelatin, and (iii) a solvent; casting the casting liquid into a mold for a microneedle; and then drying the cast solution to remove at least enough of the solvent to form the microneedle.

Embodiment 43. The method of Embodiment 42, wherein the gelatin is a fish gelatin, preferably a cold water fish gelatin.

Embodiment 44. The method of Embodiment 42, wherein the gelatin is a mammalian gelatin.

Embodiment 45. The method of any one of Embodiments 42 to 44, wherein the casting liquid comprises from 1 wt % to 10 wt % of the gelatin, preferably from 4 wt % to 8 wt % of the gelatin.

Embodiment 46. The method of any one of Embodiments 42 to 45, wherein the casting liquid further comprises one or more additional pharmaceutically acceptable excipients.

Embodiment 47. The method of Embodiment 46, wherein the one or more additional pharmaceutically acceptable excipients comprises a carbohydrate, which optionally is a disaccharide or polysaccharide.

Embodiment 48. The method of Embodiment 47, wherein the carbohydrate is selected from the group consisting of methylcellulose, maltodextrin, lactose, trehalose, sucrose, and combination thereof.

Embodiment 49. The method of any one of Embodiments 42 to 48, wherein the drying is performed with the liquid at a temperature from 4° C. to 40° C., preferably from 4° C. to 10° C.

Embodiment 50. The method of any one of Embodiments 42 to 49, wherein the casting liquid is cast into a mold defining an array of microneedles.

Embodiment 51. The method, solid composition, or microneedle of any one of embodiments 1 to 50, wherein the LNPs are stabilized by the gelatin as indicated by retaining their size range and/or polydispersity index (PDI) after being cast in solid composition form, such as from 200 nm to 400 nm and/or having a PDI a between 0 and 0.4, and/or as indicated by a transfection efficiency of a nucleic acid therapeutic agent of at least 25% after storage of the solid composition form for three months at a temperature from 2° C. to 8° C.

Modifications and variations of the methods and devices described herein will be obvious to those skilled in the art from the foregoing detailed description. Such modifications and variations are intended to come within the scope of the appended claims.

That which is claimed is:

1. A solid composition comprising:
a lipid nanoparticle (LNP) encapsulating a therapeutic agent; and
a fish gelatin.

2. The solid composition of claim 1, wherein the fish gelatin is a cold water fish gelatin.

3. The solid composition of claim 1, wherein the mass ratio of the fish gelatin to the LNP ranges from 0.5:1 to 8:1.

4. The solid composition of claim 1, wherein the mass ratio of the fish gelatin to the LNP ranges from 0.5:1 to 4:1.

5. The solid composition of claim 1, wherein the therapeutic agent comprises mRNA.

6. The solid composition of claim 1, further comprising one or more additional pharmaceutically acceptable excipients.

7. The solid composition of claim 6, wherein the one or more additional pharmaceutically acceptable excipients comprises at least one carbohydrate.

8. The solid composition of claim 7, wherein the at least one carbohydrate is selected from the group consisting of methylcellulose, maltodextrin, lactose, trehalose, sucrose, and combination thereof.

9. The solid composition of claim 7, wherein the mass ratio of the fish gelatin to the at least one carbohydrate ranges from 0.2:1 to 5:1.

10. The solid composition of claim 7, wherein the mass ratio of the fish gelatin to the at least one carbohydrate ranges from 0.5:1 to 2:1.

11. The solid composition of claim 1, wherein the solid composition in the form of a microneedle or a coating on a microneedle.

12. The solid composition of claim 1, wherein the LNP is stabilized in the solid composition for at least three months at a temperature from 2° C. to 8° C.

13. The solid composition of claim 1, wherein the LNP is stabilized in the solid composition and has at least one of, and preferably both, a size range from 200 nm to 400 nm or a polydispersity index (PDI) between 0 and 0.4.

14. The solid composition of claim 1, wherein the therapeutic agent comprises a nucleic acid, and the transfection efficiency of the nucleic acid is at least 25% after storage of the solid composition for three months at a temperature from 2° C. to 8° C.

15. A microneedle array comprising a plurality of microneedles which comprise the solid composition of claim 1.

16. A microneedle patch comprising:
at least one microneedle array of claim 15; and
a base layer cast onto a base side of the plurality of microneedles,
wherein the base layer comprises a water insoluble material.

17. A microneedle formed of a solid composition comprising:
a lipid nanoparticle (LNP) encapsulating a therapeutic agent;
a fish gelatin, wherein the mass ratio of the fish gelatin to the LNP ranges from 0.5:1 to 4:1; and
sucrose, wherein the mass ratio of the fish gelatin to the sucrose ranges from 0.5:1 to 2:1.

18. A microneedle comprising:
a solid composition which comprises:
a lipid nanoparticle (LNP) encapsulating a therapeutic agent; and
a mammalian gelatin.

19. A method of making a solid composition, the method comprising:
preparing a casting liquid which comprises (i) lipid nanoparticle (LNP) encapsulating a therapeutic agent, (ii) a fish gelatin, and (iii) a solvent;
casting the casting liquid into or onto a mold; and then
drying the cast solution to remove at least enough of the solvent to form the solid composition.

20. The method of claim 19, wherein at least one of the following conditions applies:
the drying is performed with the liquid at a temperature from 4° C. to 40° C.,
the mold defines an array of microneedles,
the fish gelatin is from 1 wt % to 10 wt % of the casting liquid,
the fish gelatin is a cold water fish gelatin,
the mass ratio of fish gelatin to LNP in the solid composition ranges from 0.5:1 to 8:1,
the therapeutic agent comprises mRNA, or
the casting liquid further comprises at least one additional pharmaceutically acceptable excipient which comprises a carbohydrate, optionally wherein the carbohydrate is selected from the group consisting of methylcellulose, maltodextrin, lactose, trehalose, sucrose, and combination thereof.

21. A method of making a microneedle, the method comprising:
preparing a casting liquid which comprises (i) lipid nanoparticle (LNP) encapsulating a therapeutic agent, (ii) a gelatin, and (iii) a solvent;
casting the casting liquid into a mold for a microneedle; and then
drying the cast solution to remove at least enough of the solvent to form the microneedle.
